(19) United States
(12) Patent Application Publication
(10) Pub. No.: US 2006/0084591 A1
(43) Pub. Date: Apr. 20, 2006

(54) Use of modulators of the signal transduction path using the protein kinase fyn for the treatment of tumorous diseases

(76) Inventors: Martin Eilers, Marburg (DE); Bernd Berwanger, Marburg (DE); Holger Christiansen, Marburg (DE); Oliver Hartmann, Berlin (DE); Helmut Schafer, Marburg (DE)

Correspondence Address: Ronald R Santucci;Frommer Lawrence & Haug, 745 Fifth Avenue, New York, NY, 10151, US

(21) Appl. No.: 10/521,514

(22) PCT Filed: Jul. 24, 2003
(86) PCT No.: PCT/EP03/08165

§ 371 Date: Aug. 30, 2005

(30) Foreign Application Priority Data
Jul. 24, 2002 (DE) 102 33 737.3

(51) Int. Cl. A61K 38/17; A61K 31/553; A61K 31/554; A61K 31/551; A61K 31/44; A61K 31/12

(52) U.S. Cl. 514/2; 514/211.09; 514/218; 514/352; 514/690; 514/211.08

(57) ABSTRACT

The present invention describes the use of modulators of the signal transduction pathway via the protein kinase Fyn (e.g. Rho-kinase inhibitors or inhibitors of the protein kinase CSK) in the treatment of tumour diseases, especially in the therapy of neuroblastoma.

The present invention describes the use of modulators of the signal transduction pathway via the protein kinase Fyn (e.g. Rho-kinase inhibitors) in the treatment of tumour diseases, especially in the therapy of neuroblastomas.

Neuroblastomas are malignant cancer conditions of the peripheral sympathetic nervous system which occur during childhood. They are one of the most frequent malignant cancer conditions of childhood. Each year about 150-200 children in Germany are afflicted by this tumour, which in advanced stages is usually incurable. The course of the disease is different in individual cases and ranges from spontaneous regression to a progressive course and the formation of metastases. The tumour develops from precursor cells of the autonomous nervous system, which controls involuntary functions such as the cardiovascular system and bowel and bladder function. In total, about 40% of children afflicted die within the first five years.

A genetic criterion used as the basis for a poor prognosis is amplification of the MYCN gene, which results in deregulated expression of the N-Myc protein in the tumour tissue. N-myc is a transcription factor which can both positively and negatively control expression of the gene. Using a cell culture model it has been shown that Myc proteins regulate both cell growth and cell proliferation.

Current clinical investigations make use of three criteria in establishing a prognosis for neuroblastoma: the tumour stage, the age of the patient, and amplification of the MYCN gene. Amplification of the MYCN gene, is, however, not a reliable criterion because tumours which do not exhibit the amplified MYCN gene can also develop.

In that context, a problem of the present invention is to utilise genetic differences in those signal transduction pathways which control proliferation and differentiation of the neuroblastoma for establishing a prognosis and for therapy in tumour diseases, especially neuroblastoma.

A problem of the present invention is to make available new methods of treating tumour diseases, especially neuroblastoma.

By means of a microarray analysis, the expression of a large number of genes in an arbitrary number of samples from many patients, all of whom are suffering from neuroblastoma, is measured in parallel. A statistical analysis is then carried out, in which clinical parameters are correlated with the expression data and consequently genes are identified which are causally involved in tumour progression. By identifying genes which are causally involved in tumour progression we open up the possibility of causal therapy for the disease.

Expression of the identified genes correlates with specific tumour stages. Surprisingly, a large proportion of those genes belong to a signal transduction pathway which proceeds via the protein kinase Fyn. This signal transduction pathway is known to regulate cell adhesion, cell proliferation and differentiation in cell cultures.

A change in cell adhesion is responsible for the formation of metastases; cell differentiation and proliferation control tumour growth itself. It is shown that the data can be validated by independent measurement methods. By means of Western blot methods it has been shown that there is a connection between the activity and/or expression of Fyn-kinase and the stage of the tumour. All the measurements show that signal transduction by Fyn is switched off in advanced tumour stages. The expression analyses underline the role of the Fyn signal transduction pathway in the formation of metastases.

In addition we demonstrate that Fyn has a causal role in the mentioned processes in neuroblastoma. Using two different neuroblastoma cell lines it is shown that the expression of Fyn, that is to say the reactivation of signal transduction, results in growth arrest, in increased adhesion and in the differentiation of neuroblastoma cells in culture. We have consequently demonstrated that the loss of signal transduction by Fyn is causal in the mentioned biological processes.

By means of a microarray analysis of human tumour samples of neuroblastoma it has been found, surprisingly, that the signal transduction pathway via the protein kinase Fyn regulates tumour growth and the formation of metastases. Furthermore, it was found that signal transduction by Fyn is switched off in the advanced tumour stage. Influencing the signal transduction pathway via Fyn-kinase, especially (re-)activation of the signal transduction pathway via Fyn, which is down-regulated in the neuroblastoma, constitutes a possibility for therapy in the treatment of neuroblastomas.

By using modulators of the signal transduction pathway via the protein kinase Fyn it is possible, in accordance with the invention, to prevent tumour growth and the formation of metastases and consequently to treat tumour diseases (especially neuroblastoma).

A further embodiment of the invention relates to modulators of the signal transduction pathway via the protein kinase Fyn, the modulators being inhibitors of enzymes which are inhibited directly or indirectly by active Fyn.

A further embodiment of the invention relates to modulators which result in an increase in Fyn activity in the tumour cells.

A further embodiment of the invention relates to modulators, the modulators being inhibitors of the protein kinase CSK, inhibitors of Rho-kinase, inhibitors of MAP-phosphatase or activators of protein kinase C.

A further embodiment of the invention relates to modulators which inhibit the proteolytic degradation of Fyn-kinase in vivo.

A further embodiment of the invention relates to modulators, the modulators being inhibitors of phosphatases that antagonise Fyn.

A further embodiment of the invention relates to modulators which contribute to an increase in signal transduction by Fyn.

In addition, the invention relates to the use of modulators of the signal transduction pathway via the protein kinase Fyn in the production of a medicament for the suppression of the formation of metastases of tumours in the early stage, it being possible for the tumour to be a paediatric or adult tumour.

The invention relates furthermore to the use of modulators as described above as a constituent of a pharmaceutical composition, characterised in that the composition, in addition to comprising the modulator as active ingredient, optionally comprises carrier substances and/or adjuvants.

A further embodiment of the invention relates to modulators as described above, characterised in that the modulator is present in the form of a pharmacologically acceptable salt, solvate, hydrate or a pharmacologically acceptable formulation.

A further embodiment of the invention relates to modulators as described above, characterised in that the modulator is present in the form of a prodrug comprising the modulator and at least one pharmacologically acceptable protecting group which is removed under physiological conditions.

A further embodiment of the invention relates to modulators as described above, characterised in that the modulator is administered orally, parenterally, rectally, by inhalation, transdermally or intranasally.

### DETAILED DESCRIPTION OF THE INVENTION

A corresponding scientific study on this subject has been published by B. Berwanger, O. Hartmann, E. Bergmann, S. Bernard, D. Nielsen, M. Krause, A. Kartal, D. Flynn, R. Wiedemeyer, M. Schwab, H. Schäfer, H. Christiansen and M. Eilers: “Loss of a FYN-regulated differentiation and growth arrest pathway in advanced stage neuroblastoma” in Cancer Cell, Vol. 2, November 2002, page 377-386, to the full content of which reference is made.

In order to gain an insight into the molecular sequence of the development of neuroblastomas, expression profiles of 94 individual tumour tissue samples were produced, using a human Unigene 4608 cDNA chip. Each chip is hybridised with cDNA as reference which is derived from a human neuroblastoma cell line (SHEP). The tumours were so selected that they reflected the distribution of the tumour stages and the MYCN amplification of the tumour bank as a whole. In order to allow comparisons between the individual spots and the arrays, each signal was corrected in relation to its background, and the log₂-transformed on/off regulation intensity ratios were calculated and standardised. A two-sample t statistic with adjusted p values was used in order to identify differently expressed genes (Callow et al., 20⁰0). The adjusted p values simultaneously correct the testing of 4608 genes and estimate the overall probability of detecting an incorrect gene (see methods).

We found 123 differently expressed genes when we compared MYCN-amplified (n=17) and non-amplified (n=77) tumours (adjusted p value<0.05). These genes were used in a non-controlled, hierarchical cluster, and the analysis showed that all tumours except three were correctly assigned to one of the two classes. Functional annotation showed that most of the genes that were switched on in MYCN-amplified tumours encode proteins which play a role in protein synthesis, in metabolism and in regulation of the cell cycle; this agrees with the microarray data produced by using inducible systems in cell culture. These results show that Myc proteins regulate both cell growth and also cell proliferation and that the models developed using the cell culture extend to the formation of the neuroblastoma. The expression of a series of genes which were identified as target genes of (c-)Myc in cell culture experiments was significantly different between the two classes of tumours. A large group of genes most of which were switched off in MYCN-amplified tumours encode proteins which are involved in multi-stage signal transduction pathways. This shows that Myc proteins constitute a negative feedback signal for signal transduction pathways. Surprisingly, the cell cycle genes which increasingly appear in MYCN-amplified tumours encode proteins which are known to function as a checkpoint response or in the G2 or M phase of the cycle, which points to a new function of Myc in checkpoint control and late in the cell cycle.

The deregulation of those genes can, in simple manner, reflect the advanced tumour stage of most MYCN-amplified tumours (Table 1). In order to exclude that possibility, we compared their expression between stage 4 of MYCN-amplified and non-amplified tumours. The expression of all genes analysed by us was regulated by MYCN amplification, irrespective of the tumour stage. Conversely, the deregulation could reflect direct regulation by N-Myc. This interpretation is in agreement with the fact that many E-boxes are present in the promoter and introns of the MAD2, CENPE and AURORA2 genes. (FIG. 1, Field e). Chromatin immunoprecipitation did indeed show that N-Myc is bound in vivo to the E-boxes of MAD2 genes. From that we concluded that at least some of the genes identified by us are direct target genes of N-Myc.

TABLE 1
Table 1: Clinical parameters of 94 patients taking part in this
study. Age and stage are highly disparate, and because of the
low number they could not be analysed independently.

| | | STAGE | | | | | |
|---|---|---|---|---|---|---|---|
| | | 1 | 2 | 3 | 4 | 4S | Total |
| MYCN | Non- | 19 | 8 | 17 | 21 | 12 | 77 |
| | amplified | | | | | | |
| | Amplified | 1 | 1 | 4 | 8 | 3 | 17 |
| Age | <12 months | 18 | 1 | 11 | 5 | 15 | 50 |
| | >12 months | 2 | 8 | 10 | 24 | 0 | 44 |
| Age | Mean | 4.3 | 23.4 | 28.7 | 42.6 | 4.0 | 23.3 |
| | (months) | | | | | | |
| | Standard | 4.2 | 17.8 | 41.1 | 33.6 | 3.4 | 31.6 |
| | deviation | | | | | | |
| | (months) | | | | | | |
| Total | | 20 | 9 | 21 | 29 | 15 | 94 |

An initial study showed that, among the non-MYCN-amplified tumours, stages 1 and 4, but not stages 2, 3 and 4S, show differences in their expression profiles (data not shown). We found 36 indicative genes which were differently expressed in tumours in stage 1 (n=19) and in stage 4 (n=21) (adjusted p value<0.2). That set of genes showed a slight overlap with the group of genes which differentiates MYCN-amplified tumours from non-amplified tumours; functional annotation of the genes showed that the genes which encode proteins which play a role in metabolism and protein synthesis were apparently absent from the group of stage-specific genes, in contrast to the genes which were characteristic of MYCN-amplified tumours. In contrast, a characteristic percentage of genes which were differently expressed encodes genes which are involved in signal transfer by the non-receptor tyrosine kinase Fyn and the actin cytoskeleton; these genes were down-regulated in co-ordinated manner in the advanced stage of the neuroblastoma. This group includes Fyn itself, actin filament binding protein (AFAP), a protein which binds to Src and Fyn-kinase and activates the latter; α-catenin (CTNNA1), an actin-binding protein whose binding to β- and γ-catenin is regulated by Fyn-dependent phosphorylation; the neural cell adhesion protein NRCAM, which signals by way of non-receptor tyrosine kinases and the actin-binding proteins tropomodulin and MARCKS.

Western blot confirmed the reduced expression of Fyn in tumours of stage 4 compared to stage 1; in addition, the experiment showed, in agreement therewith, slower migration of the Fyn protein in extracts from all tumours of stage 1 compared to those of stage 4, which shows that the autophosphorylated (active) form is present. Phosphatase treatment confirmed that the differing migration came about as a result of the phosphorylation. In conformity with those observations, a high Fyn-kinase activity was obtained in tissue samples from tumours of stage 1, for which immunocomplex kinase assays were used which measure both autophosphorylation and also phosphorylation of the exogenous substrate, enolase (Wolf et al., 2001). In contrast, the Fyn-kinase activity in extracts from tumours of stage 4 was variable and, on average, substantially lower.

In order to test whether Fyn plays a role in differentiation and regulation of cell proliferation of neuroblastoma cells, we used a transient transfection in order to express Fyn in SH-SY5Y cells—a human neuroblastoma cell line derived from a stage 4 tumour (Pahlman et al., 1981). Expression of wild-type Fyn induced the extension of multiple neurites and overt morphological characteristics of differentiation. Staining with antibodies directed against cyclin A as a marker protein of cell proliferation showed that the cells which expressed active Fyn had exited from the cell cycle. In contrast, cells which expressed a kinase-negative allele (FynK299M) showed no signs of morphological differentiation. In conformity with the role of AFAP in the activation of Fyn, the expression of a constitutively active allele of AFAP induced morphological changes highly reminiscent of active Fyn, whereas a dominant negative allele of AFAP does not influence differentiation.

We repeated the experiments in IMR-32 cells, a human neuroblastoma cell line that carries an amplified MYCN gene (Clementi et al., 1986). Similarly to the results obtained in SH-SY5Y cells, the expression of active Fyn-kinase induced neurite extension and cell cycle exit. This shows that the induction of differentiation by Fyn occurs even in the presence of an amplified MYCN gene. This agrees with the finding that the expression of FYN, AFAP, NRCAM and CTNNA1 is down-regulated equally in MYCN-amplified tumours and in non-amplified tumours, based on tumours in stage 1.

In all, we identified two genetic programs which regulate the development of neuroblastomas, one which is controlled by amplification of the MYCN gene and a second, stage-specific program of gene expression. The two programs are, to a high degree, independent of one another because (a) the two groups of genes show little overlap, (b) the genes are down-regulated by MYCN amplification irrespective of tumour stage and (c) the tumour-stage-specific genes are down-regulated irrespective of MYCN amplification. The deregulated expression of MYCN activates genes encoding proteins which are involved both in advancement of the cell cycle and also in cell growth and suppresses genes encoding proteins involved in multi-stage signal processes in a human tumour. These findings show, specifically, that Myc proteins control gene expression in the G2 phase of the cell cycle and activate genes which are involved in checkpoint processes.

Our data show that Fyn-kinase regulates the proliferation and differentiation of neuroblastoma cells in vivo; this is likewise supported by the finding that the stage-specific expression profile is predictive of survival. Detailed expression profiles of individual tumour stages showed that the down-regulation of Fyn is most marked between stages 1 and 2, which correlates with the formation of metastases in the local lymph nodes. The down-regulation of Fyn and also the modified cell adhesion control the formation of local metastases in vivo.

Active Fyn can perform its function in a number of ways: In neuronal cells, non-receptor tyrosine kinases phosphorylate Rho-GAP, which results in inactivation of Rho and induction of differentiation. It was found that there are molecules in signal transduction pathways which are inhibited by Fyn signalling, which makes them into candidates for a therapeutic intervention. In accordance with the invention, influencing the signal transduction pathway downstream of Fyn provides a therapeutic approach for advanced-stage neuroblastomas.

Preferred target molecules in the Fyn signal transduction pathway are those which are inhibited by Fyn signal transduction.

Preferred modulators are inhibitors of enzymes which are directly or indirectly inhibited by active Fyn.

Further preference is given to modulators which result in an increase in Fyn activity in tumour cells (especially in neuroblastomas).

Special preference is given to inhibitors of the protein kinase CSK, which is expressed in neuroblastomas and is a negative regulator of Fyn in vivo.

Preference is moreover given to modulators which inhibit the proteolytic degradation of Fyn-kinase in vivo, e.g. general inhibitors of the proteasome (LLNL, MG132) or specific inhibitors of the E3 ligases involved.

Further preferred modulators are inhibitors of phosphatases which antagonise Fyn, e.g. MAP-kinase phosphatase 1.

Preference is also given to modulators which contribute to an increase in signal transduction by Fyn, e.g. Rho-kinase inhibitors, MAP-phosphatase inhibitors or activators of protein kinase C.

The present invention furthermore encompasses pharmacologically acceptable salts, solvates, hydrates or pharmacologically acceptable formulations of the described modulators.

Examples of pharmacologically acceptable salts are salts of physiologically acceptable mineral acids such as hydrochloric acid, sulphuric acid and phosphoric acid or salts of organic acids such as methanesulphonic acid, p-toluenesulphonic acid, lactic acid, acetic acid, trifluoroacetic acid, citric acid, succinic acid, fumaric acid, maleic acid and salicylic acid. The modulators according to the invention may be solvated, especially hydrated. The hydration may occur, for example, during the preparation process or as a consequence of the hygroscopic nature of the initially anhydrous compounds. When the described modulators contain asymmetric C atoms, they may be present either in the form of mixtures of diastereomers, mixtures of enantiomers or in the form of optically pure compounds.

The pharmaceutical compositions according to the present invention comprise at least one of the described modulators as active ingredient and, optionally, carrier substances and/or adjuvants.

The prodrugs, to which the present invention also relates, consist of a modulator according to the invention and at least one pharmacologically acceptable protecting group which is removed under physiological conditions, for example an alkoxy, aralkyloxy, acyl or acyloxy group, e.g. an ethoxy, benzyloxy, acetyl or acetoxy group.

The present invention relates also to the use of the modulators in the production of medicaments for the prevention and/or treatment of tumour diseases (especially neuroblastoma). In general, the modulators are administered either individually or in combination with any other desired therapeutic agent, using the known and acceptable methods. Such therapeutically useful agents can be administered by one of the following routes: orally, for example in the form of dragées, coated tablets, pills, Semi-solid substances, soft or hard capsules, solutions, emulsions or suspensions; parenterally, for example in the form of an injectable solution; rectally in the form of suppositories; by inhalation, for example in the form of a powder formulation or spray, transdermally or intranasally. For the production of such tablets, pills, semi-solid substances, coated tablets, dragées and hard gelatin capsules, the therapeutically usable product can be mixed with pharmacologically inert, inorganic or organic pharmaceutical carrier substances, for example with lactose, sucrose, glucose, gelatin, malt, silica gel, starch or derivatives thereof, talcum, stearic acid or salts thereof, skimmed milk powder and the like. For the production of soft capsules, pharmaceutical carrier substances such as, for example, vegetable oils, petroleum, animal or synthetic oils, waxes, fats and polyols can be used. For the production of liquid solutions and syrups, pharmaceutical carrier substances such as, for example, water, alcohols, aqueous saline solution, aqueous dextrose, polyols, glycerol, vegetable oils, petroleum and animal or synthetic oils can be used. For suppositories, pharmaceutical carrier substances such as, for example, vegetable oils, petroleum, animal or synthetic oils, wax, fat and polyols can be used. For aerosol formulations, compressed gases that are suitable for the purpose can be used, such as, for example, oxygen, nitrogen and carbon dioxide. The pharmaceutically acceptable agents may also comprise additives for preserving and stabilising, emulsifiers, sweeteners, flavourings, salts for altering the osmotic pressure, buffers, encapsulation additives and anti-oxidants.

Combinations with other therapeutic agents can include other active ingredients which are customarily used in the treatment of tumour diseases (especially neuroblastoma).

For the prevention and/or treatment of the diseases described above, the dose of the modulators according to the invention can vary within wide limits and can be adjusted to individual requirements. In general, a dose of from 0.1 μg to 100 mg/kg of body weight per day is suitable, a preferred dose being from 0.5 to 10 mg/kg per day. In suitable cases, the dose may also be below or above the stated values.

### EXAMPLES

Examples of Rho-kinase inhibitors are described in EP0370498, U.S. Pat. No. 4,997,834, EP0956865, U.S. Pat. No. 6,218,410, U.S. Pat. No. 4,678,783, U.S. Pat. No. 6,153,608, EP0885888, WO0168607 and WO0156988. Specifically, there may be mentioned herein compounds I (fasudil), II (hydroxyfasudil), III and IV:

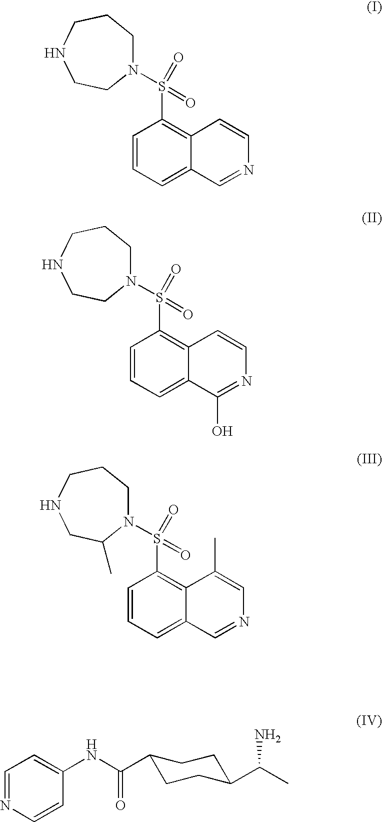

Examples of activators of protein kinase C are compound V, the compound EP-70905 of Europeptides, the natural substances bryostatin, teleocidin, aplysiatoxin and also esters of phorbol and ingenol. Further compounds such as VI and VII are described in J. D. Winkler et al. J. Am. Chem. Soc. 1999, 121, 296-300.

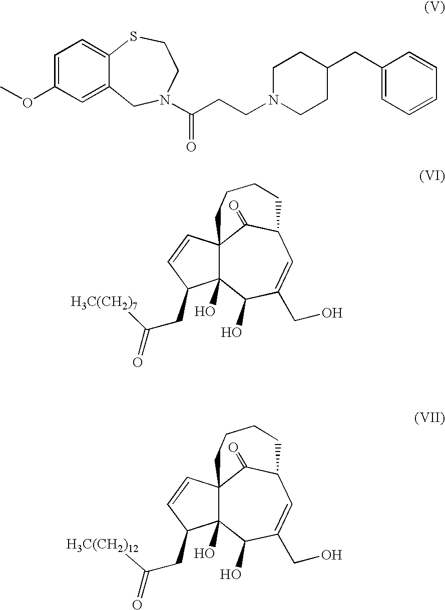

An example of an MAP-kinase phosphatase 1 inhibitor is the compound MX 7091 of Maxima Pharmaceuticals.

An example of a CSK inhibitor is the natural substance staurosporine.

Materials and Methods

Microarray Experiments

The chip used comprises the cDNA set gf200 of Research Genetics (http://www.resgen.com) and, in addition, 100 cDNAs that had already been described previously as potentially suitable for the prognosis of neuroblastoma development (for details see http://www.imt.uni-marburg.de). Each cDNA was spotted twice per chip. The chips were produced as described in Hegde et al., 2000, using a GMS 417 arrayer.

An initial histochemical study of a series of 100 randomly selected tumours revealed that approximately 95% of the tumours contained less than 5% non-tumour cells in the tissue samples (see Bergmann et al., 2001). Therefore, no further attempt was made to dissect the tumour tissue before preparation of the RNA. Total RNA from the neuroblastoma tissue and SHEP was isolated using a Qiagen RNA isolation kit in accordance with the manufacturer's instructions. 40 μg of total RNA were used in order to produce Cy3 and Cy5 fluorescently labelled cDNA in accordance with the published protocol (http://brownlab.stanford.edu). The chips were scanned using a GMS 418 fluorescence scanner, and the images were analysed using IMAGENE 3.0 software. The expression data were confirmed either by Northern blot analysis or by real-time RT-PCR assays.

Standardisation and Quality Control

ImaGene 3.0: The software parameters such as “signal ranges” or “spot detection threshold” (for details see the ImaGene user manual) were optimised for maximum reproducibility prior to image analysis in our experiment. For each spot the median signal and the background intensities for both channels are obtained. In order to determine the differences between spots, the corrected background ratio of the two channels was calculated and log₂-transformed. In order to balance the fluorescence intensities of the two dyes and also to allow a comparison of the expression level across experiments, the raw data were standardised. First, we used a pin-wise intensity-dependent standardisation (Yang et al., 2002) in order to correct for inherent bias on each chip (the lowess scatter-plot smoother). In a second step, a general standardisation was performed in order to centre the log-ratios for each array at 0 (in order to take into account general staining and scanner effects). Because each gene was spotted on the chip twice, the mean log-ratios M were calculated from the replicates. If the replicates differed by more than a factor of four or if the background intensity was higher than the signal intensity, that gene was excluded from the array.

Statistical Analysis

The final data matrix consisted of 4608 standardised gene expression measurements (log₂-ratios) from 94 individual tumours (with missing values). In order to compare the expression profile between two independent groups, a two-sample t statistic was used for each gene. In order to account for multiple testing, we calculated the adjusted p values for each gene, in the course of which we used a step-down permutation algorithm (Westfall and Young, 1993, algorithm 4.1). This strategy had been applied to microarrays before (Callow et al., 2000). The permutation algorithm provides a powerful check on the family-wise error rate (FWER) and takes into account correlation of the variables (genes). The procedure does not rely upon a normality assumption; it is assumed that the t statistic has asymptotically the same null distribution for all genes (or that the p values are monotonic in the observed t statistics across the genes).

Cluster Analysis

Prior to cluster analysis, the expression profile of each gene was centred by subtraction of the mean observed value. Average linkage hierarchical clustering was then performed for genes and also for chips, in the course of which the Euclidean distance metric as implemented in the program J-Express was used (Dysvik and Jonassen, 2001).

Western Blots, Immunoprecipitation, Phosphatase Treatment

The following antibodies were used in Western blots, immunofluorescence experiments and immunoprecipitations: α-Fyn: (sc-434); α-cdk2 (sc-163), α-cyclinA (sc-751) all from Santa Cruz. Neuroblastoma tissue was lysed as described (Bergmann et al., 2001). For the phosphatase treatment, 500 μg of cellular proteins were incubated overnight at 4° C. with 5 μg of Fyn-antibody bound to protein G beads. Immunocomplexes were incubated either with λ-protein phosphatase (NEB) or with λ-protein phosphatase and phosphatase inhibitors. Immunocomplexes were electrophoretically separated by means of a 10% SDS PAGE, transferred to a PVDF membrane and detected with an α-Fyn antibody.

In Vitro Kinase Assays

500 μg of cellular proteins were immunoprecipitated with 5 μg of α-Fyn antibody bound to protein G beads, washed and equilibrated in kinase assay buffer, incubated for 15 minutes with 10 μCi γ-ATP (Amersham) and 0.125 mg/ml of enolase, electrophoretically separated by means of a 10% SDS PAGE and dried. The results were visualised on a Fuji Phosphorimager and quantified using Bildeich software.

Chromatin Immunoprecipitation

Chromatin immunoprecipitation was performed as previously described (Bouchard et al., 2001). Nuclear extracts were immunoprecipitated overnight at 4° C. with 3 μg of α-N-Myc antibody or control antibody bound to protein A and protein G beads. For PCR analysis, a specific primer pair for intron 1 of prothymosin alpha (as positive control) and also primer pairs amplifying the indicated regions of the MAD2 gene were used. Primer sequences are available upon request.

Cell Culture Experiments

SH-SY5Y and IMR-32 neuroblastoma cell lines were cultured in RPMI 1640 supplemented with 10% heat-inactivated FCS. CMV-driven expression constructs encoding wild-type Fyn (Fynwt) and FynK299M have been described (Wolf et al., 2001). Plasmids encoding AFAPALZ and AFAPA180-226 have been described (Baisden et al., 2001). For transient transfections, the cells were first allowed to grow for 6 hours on cover slips coated with a 1:5 dilution of Matrigel (Becton-Dickinson). Transfection was performed by using 5 μg of DNA and also a Lipofectin reagent (Invitrogen). The cells were fixed with paraformaldehyde after 60 hours, washed and stained with a monoclonal α-Fyn antibody (Santa Cruz) or an α-AFAP rabbit polyclonal antibody (Baisden et al., 2001).

### LITERATURE

Baisden, J. M., Qian, Y., Zot, H. M., and Flynn, D. C. (2001). The actin filament-associated protein AFAP-110 is an adaptor protein that modulates changes in actin-filament integrity. Oncogene 20, 6435-47.
Bergmann, E., Wanzel, M., Weber, A., Shin, I., Christiansen, H., and Eilers, M. (2001) Expression of P27(KIP1) is prognostic and independent of MYCN amplification in human neuroblastoma, Int. J. Cancer 95, 176-83.
Bouchard, C., Dittrich, O., Kiermaier, A., Dohmann, K., Menkel, A., Eilers, M., and Luscher B. (2001). Regulation of cyclin D2 gene expression by the Myc/Max/Mad network: Myc-dependent TRRAP recruitment and histone acetylation at the cyclin D2 promoter. Genes Development 15, 2042-7.
Callow, M. J., Dudoit, S., Gong, E. L., Speed, T. P., and Rubin, E. M. (2000). Microarray expression profiling identifies genes with altered expression in HDL-deficient mice. Genome Research 10, 2022-9.
Clementi, F., Cabrini, D., Gotti, C., and Sher, E. (1986) Pharmacological characterization of cholinergic receptors in a human neuroblastoma cell line. J. Neurochem 47, 291-7.
Dysvik, B., and Jonassen, I. (2001) J-Express: exploring gene expression data using Java. Bioinformatics 17, 369-370.
Hegde, P., Qi, R., Abernathly, K., Gay, C., Dharap, S., Gaspard, R., Hughes, J. E., Snesrud, E., Lee, N., and Quackenbusch, J. (2000). A concise guide to cDNA microarray analysis. Biotechniques 29, 548-50, 552-4, 556 passim.
Pahlmann, S., Odelstad, L., Larsson, E., Grotte, G., and Nilsson, K., (1981). Phenotypic changes of human neuroblastoma cells in culture induced by 12-O-tetradodecanoyl-phorbol-13-acetate. Int J Cancer 28, 583-9.
Westfall, P. H., and Young, S. S. (1993) Resampling-Based Multiple Testing. Examples and Methods for p-Value Adjustment.
Wolf, R. M., Wilkes, J. J., Chao, M. V., and Resh, M. D., (2001). Tyrosine phosphorylation of p190 RhoGAP by Fyn regulates oligodendrocyte differentiation. J. Neurobiol. 49, 62-78.
Yang, Y. H., Dudoit, S., Luu, P., Lin, D. M., Peng, V., Hgai, J., and Speed, T. P. (2002). Normalization for cDNA microarray data: a robust composite method addressing single and multiple slide systematic variation. Nucleic Acids Research 30, e15.

1. Use of modulators of the signal transduction pathway via the protein kinase Fyn for the prophylaxis and/or treatment of tumour diseases.

2. Use of modulators according to claim 1 in the treatment of neuroblastomas.

3. Use according to claim 1, the modulators being inhibitors of enzymes that are directly or indirectly inhibited by active Fyn.

4. Use of modulators according to claim 1 which result in an increase in Fyn activity in the tumour cells.

5. Use of modulators according to claim 1, the modulators being inhibitors of the protein kinase CSK.

6. Use of modulators according to claim 1, the modulators being inhibitors of Rho-kinase.

7. Use of modulators according to claim 1, the modulators being inhibitors of MAP-phosphatase.

8. Use of modulators according to claim 1, the modulators being activators of protein kinase C.

9. Use of modulators according to claim 1 which inhibit the proteolytic degradation of Fyn-kinase in vivo.

10. Use of modulators according to claim 1, the modulators being inhibitors of phosphatases that antagonise Fyn.

11. Use of modulators according to claim 1 which contribute to an increase in signal transduction by Fyn.

12. Use of modulators of the signal transduction pathway via the protein kinase Fyn in the production of a medicament for the suppression of the formation of metastases of tumours in the early stage.

13. Use of modulators according to claim 9, the tumour being a paediatric or adult tumour.

14. Use of modulators according to claim 1 as a constituent of a pharmaceutical composition, wherein the composition, in addition to comprising the modulator as active ingredient, optionally comprises carrier substances and/or adjuvants.

15. Use of modulators according to claim 1, wherein the modulator is present in the form of a pharmacologically acceptable salt, solvate, hydrate or a pharmacologically acceptable formulation.

16. Use of modulators according to claim 1, wherein the modulator is present in the form of a prodrug comprising the modulator and at least one pharmacologically acceptable protecting group which is removed under physiological conditions.

17. Use of modulators according to claim 1, wherein the modulator is administered orally, parenterally, rectally, by inhalation, transdermally or intranasally.
